(19) United States
(12) Patent Application Publication
(10) Pub. No.: US 2011/0251124 A1
(43) Pub. Date: Oct. 13, 2011

(54) REGULATION OF MINERAL AND SKELETAL METABOLISM

(75) Inventors: Thomas Haberberger, Hayward, CA (US); David Rosen, Hayward, CA (US); Yoshinari Kumagai, Hayward, CA (US)

(73) Assignee: Acologix, Inc.

(21) Appl. No.: 12/967,888

(22) Filed: Dec. 14, 2010

Related U.S. Application Data
(63) Continuation of application No. 11/926,460, filed on Oct. 29, 2007, now abandoned.
Application No. 11/926,460 is a continuation of application No. 11/466,673, filed on Aug. 23, 2006, now abandoned.
(60) Provisional application No. 60/713,154, filed on Aug. 30, 2005.
(60) Provisional application No. 60/717,115, filed on Sep. 13, 2005.
(60) Provisional application No. 60/807,797, filed on Jul. 19, 2006.

(51) Int. Cl. A61K 38/17; A61P 5/18; A61P 19/10

(52) U.S. Cl. 514/11.8; 514/16.9

(57) ABSTRACT

A method is disclosed whereby levels of calcium, phosphate and parathyroid hormone are measured in a patient. The patient is treated with a formulation comprising a compound having phosphotonin activity and thereafter measurements are made again. Dosing of the formulation is adjusted based on measurements with measuring, administering and adjusting dosing continually repeated as needed.

### CROSS REFERENCES

This application claims the benefit of U.S. Provisional Application Nos. 60/713,154 filed Aug. 30, 2005; 60/717,115 filed Sep. 13, 2005; and 60/807,797 filed Jul. 19, 2006 which applications are incorporated herein by reference.

### FIELD OF THE INVENTION

The present invention relates to a method of treatment which involves the regulation of metabolisms and biological functions that are affected by the hormonal effects of parathyroid hormone (PTH), including, but not limited to, the formation, destruction, and turnover of the skeletal tissues. More specifically, the present invention relates to a method to control the metabolism of parathyroid hormone (PTH).

### BACKGROUND OF THE INVENTION

PTH is an endocrine hormone produced by parathyroid glands and circulated systemically to play key roles in mammals. In collaboration with a few other hormones such as calcitriol which is an active form of vitamin D₃, calcitonin, and PTHrp, PTH has been known to regulate both systemic and local metabolisms of Calcium (Ca) and phosphate (PO₄).

Ca and PO₄ play central roles in many of the basic processes essential to the biological and physiological functions of various cells and the mineralization of the skeletal tissues such as bone, cartilage, and teeth. In particular, skeletal mineralization is dependent on the regulation of Ca and PO₄ in the body and any disturbances in Ca—PO₄ homeostasis can have severe repercussions for several important tissues including, the kidney, vasculature, and on the integrity of the hard tissues.

In the kidney, both Ca and PO₄ are lost passively into the glomerular filtrate and actively reabsorbed in distal and proximal tubules to maintain their physiological levels in the body fluid such as blood. In the intestine, both Ca and PO₄ are absorbed from foods and regulation of such absorption is also known to contribute to the systemic homeostasis of Ca and PO₄. Skeletal tissues, particularly bones, are also known to be one of the organs that play important roles in Ca and PO₄ homeostasis. Skeletal tissues store or release Ca and PO₄ to maintain their adequate levels in the circulation.

Among the few hormones that are related to Ca and PO₄ metabolisms, PTH is known to be the most influential hormone not only on the homeostasis of Ca and PO₄ but also bone turnover. PTH increases the active reabsorption of Ca in the renal tubule thereby increasing circulating Ca. Further, PTH is able to inhibit the active reabsorption of PO₄ in the renal tubule thereby decreasing the circulating PO₄. PTH increases the skeletal tissue turnover and increases or decreases the bone mass depending upon the microenvironment. Generally, continuous exposure of bones to PTH results in a higher bone resorption, which recruits more Ca into the circulation from the bone.

PTH is produced and secreted into the circulation by parathyroid glands. Parathyroid glands are sensitive to the serum levels of Ca and PO₄ to regulate their secretion of PTH. For instance, low serum Ca levels or high serum PO₄ levels increase PTH secretion and high serum Ca levels or low serum PO₄ levels decrease it. Ca sensing molecule (Ca sensor or Ca receptor) has been cloned and its agonists and antagonists have been synthesized to therapeutically regulate the PTH secretion levels by the parathyroid glands.

Calcitonin is produced by the thyroid glands and inhibits osteoclast functions, which thereby reduces bone resorption. As a result, more Ca is retained in the bone without entering the circulation.

Calcitriol stimulates Ca absorption in the intestine from the food to increase its circulating levels. Calcitriol also effects bone turnover and reduces PTH secretion.

In addition to these hormones known for decades, a few newly identified molecules such as matrix extracellular phosphoglycoprotein (MEPE; Genomics 67 54 2000, Bone 34 303-319 2004), fibroblast growth factor-23 (FGF-23; JCEM 86 497-500 2001), and frizzled related protein-4 (FRP-4; Current Opinion in Nephrology and Hypertension 11 423-430 2002) are claimed as “phosphatonin” which selectively regulates the serum levels of PO₄. It is believed that those “phosphatonin” molecules reduce the active PO₄ reabsorption of renal tubules by suppressing sodium (Na⁺) dependent phosphate cotransporter (NaPi or NPT; Hilfiker, PNAS 95(24) (1998), 14564-14569). The sodium (Na⁺) dependent phosphate cotransporter is believed to be the molecule most responsible for active PO₄ transport in the renal tubule and intestine.

These molecules having phosphatonin activities were identified by observing the clinical symptoms of patients suffering from rare diseases such as X-linked hypophosphatemic rickets (XLH), autosomal dominant rickets (ADR), and tumor induced osteomalacia (TIO) [or alternatively called oncogenic hypophosphatemic osteomalacia (OHO)]. These diseases share very similar symptoms such as hypophosphatemia (extraordinarily low serum PO₄ levels), phosphaturia (excessive leakage of PO₄ into the urine), extremely low levels of calcitriol in the circulation, and osteomalacia although the serum levels of Ca and PTH are within the normal range.

The published biological data of MEPE, FGF-23, and FRP-4 have thus far suggested that their biological activities were selective to PO₄ and calcitriol (Bone 34 303-319 2004; Am J Physiol Renal Physiol. 2005 February; 288(2):F363-70; J Clin Invest. 2003 September; 112(5):785-94.)

Disorder of Ca and PO₄ homeostasis and imbalance of the mineral metabolism hormones such as PTH and calcitriol are typically observed in chronic kidney disease. They are broadly recognized as the pathogens of several severe secondary complications such as vascular calcification which commonly results in heart failure, cerebrovascular disorders, even acceleration of the disease progression, and renal osteodystrophy that is a severe bone loss associated with the chronic kidney disease.

Chronic kidney disease usually takes years to progress toward the end stage renal disease (ESRD) where patients require dialysis or kidney transplantation in order to stay alive. In the process of the disease progression, imbalance of Ca, PO₄, and PTH gradually advances. For the declining filtering functions by kidneys, serum levels of PO₄ tend to elevate initially. To prevent such PO₄ elevation, more PTH is secreted because PTH has inhibitory activities on PO₄ reabsorption at renal tubules. This is generally well known as secondary hyperparathyroidism. Once the elevated levels of PTH become insufficient to prevent serum PO₄ elevation, serum PO₄ levels start to elevate significantly (hyperphosphatemia). Along with this process, higher PTH increases renal reabsorption of Ca and bone resorption, which pushes up serum Ca levels. As a result of all of these events, chronic kidney disease patients typically demonstrate hyperphosphatemia, high serum calcium-phosphorus (Ca.P) product, hyperparathyroidism, and renal osteodystrophy.

Thus, normalizing serum levels of Ca, PO₄, and PTH as well as treating the impaired skeletal metabolism (i.e., renal osteodystrophy) are the clinical needs in these patients.

To address the complicated Ca, PO₄, and PTH imbalance in chronic kidney disease patients, several therapeutic compounds have been developed and used. “Phosphate binders” such as calcium carbonate, calcium acetate (PhosLo), cevelamar chloride (Renagel®), and lanthanum carbonate (Fosrenol) were developed to control hyperphosphatemia. However, these drugs simply bind PO₄ in the food in intestine before they are absorbed into the bloodstream. Although they do offer some degree of effect, compliance is low due to the large volume of pills that need to be taken with each meal at least for several weeks. Even if the patients are compliant, the reduction in serum phosphate levels are generally marginal.

A few therapeutics to control PTH have been developed or are under development. Calcium receptor agonist such as Cinacalcet binds calcium receptor on parathyroid gland and reduce production and secretion of PTH. However, calcium agonists are not effective for the reduction of serum phosphate.

Vitamin D₃ and its derivatives are widely used in chronic kidney disease patients to address the same problems. However, they sometimes stimulate Ca absorption in the intestine and their excessive use sometimes causes a dynamic bone disease where bone turnover is almost totally shut down and the bone cannot be remodeled.

Thus a therapeutic that could address both phosphate and calcium levels while reducing PTH levels would be a unique and highly desirable therapy for a wide range of patients including those with chronic kidney disease.

### BRIEF DESCRIPTION ABOUT THE DRAWINGS

FIG. 1 indicates the plasma concentration of recombinant human MEPE (rhMEPE) made by E. coli or CHO cells at different time points after a single injection to rats.

FIG. 2 demonstrates the plasma levels of phosphate normalized with creatinine in mice at different time points when rhMEPE was intraperiotoneously injected to the mice with different administration schedule.

FIG. 3 shows the plasma levels of parathyroid horomen (PTH) in mice at different time points when rhMEPE was intraperitoneously injected to the mice with different administration schedule.

FIG. 4 indicates the plasma concentration of MEPE at different time points up to about 8 hours after a single injection of pegylated rhMEPE (PEG-MEPE) to rats.

FIG. 5 shows the plasma concentration of PEG-MEPE at a 24 hour time point after a single injection to mice.

FIG. 6 demonstrates the plasma concentration of intact parathyroid hormone (iPTH) in mice at 24 hour time point after a single injection of PEG-MEPE to mice.

FIG. 7 exhibits the plasma concentration of MEPE at a 72 hour time point after a single injection of PEG-MEPE to mice.

FIG. 8 indicates the plasma concentration of iPTH in mice at a 72 hour time point after a single injection of PEG-MEPE to mice.

FIG. 9 exhibits the plasma levels of calcium normalized with creatinine in mice at different time points when rhMEPE was intraperiotoneously injected to the mice with different administration schedule.

### DETAILED DESCRIPTION OF THE PRESENT INVENTION

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a peptide” includes a plurality of such “peptides” and reference to “a body fluid” includes reference to one or more body fluids and equivalents thereof known to those skilled in the art and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human and includes: (a) preventing the disease from occurring in a subject which may be predisposed to the disease but has not yet been diagnosed as having it; (b) inhibiting the disease, i.e., arresting its development; or (c) relieving the disease, ilel, causing regression of the disease. The present invention is directed towards treating patients with medical conditions relating to a disorder of phosphate metabolism. Accordingly, a treatment of the invention would involve preventing, inhibiting or relieving any medical condition related to calcium, phosphate, or PTH disorders.

Methods of treatment of the invention include treating rare diseases such as X-linked hypophosphatemic rickets (XLH), autosomal dominant rickets (ADR), and tumor induced osteomalacia (TIO) which is also referred to as oncogenic hypophosphatemic osteomalacia (OHO). Methods of the invention include treating various forms of hypophosphatemia associated with extremely low serum PO₄ levels and treating phosphaturia associated with excessive leakage of PO₄ into the urine. Methods include treating extremely low levels of calcitrol in the circulation and to treating osteomalacia although the serum levels of Ca and PTH are within normal ranges.

Treatment in accordance with the invention can include monitoring, measuring, and/or determining in any manner the level of any or all of Ca, PO₄ and PTH and thereafter administering the formulation of the invention and may further include thereafter again measuring, monitoring and determining levels or all or any of Ca, PO₄ and PTH and thereafter readministering the formulation in the same amount and/or adjusting the amount based on the remeasured level so as to determine the effect of the first administration of all or any of the levels and thereby adjusting dosing accordingly. The method steps of measuring and administering described here can be repeated as needed over a period of days, weeks, months or years. The measurement may be on blood, urine, or any body fluid or tissue.

By “therapeutically effective amount” is meant an amount which relieves to some extent one or more symptoms of a disease or disorder in the patient; or returns to normal either partially or completely one or more physiological or biochemical parameters associated with or causative of the disease or disorder. Thus, a therapeutically effective amount can be an amount effective to prophylactically decrease the likelihood of the onset of a disease or disorder. A therapeutically effective amount may be an amount which shows to have a therapeutically meaningful effect on levels of Ca, PO₄ and/or PTH after measuring prior to administration and measuring after administration.

### Invention in General

The present invention relates to a method to control the metabolism of parathyroid hormone (PTH) in a manner which is totally distinctive from the currently understood physiological mechanisms. In one of the particular embodiment of the present invention, a new method to control the circulating levels of PTH is presented. In accordance with an embodiment of the invention a formulation is comprised of a carrier and a peptide chosen from SEQ ID NO:2, 3, 5, 6, 8-13 and any biologically operable fraction thereof comprised of at least 51 amino acids. In another embodiment the patient's body fluids (e.g. serum and/or urine) are tested to determine levels of all or any of Ca, PO₄ and PTH. The formulation is administered and after an appropriate period of time the patient's body fluids are again tested with respect to levels of all or any of Ca, PO₄ and PTH. Adjustments in dosing may be required after determining levels obtained after initial treatment. Treatment then continues with repeated administration of the formulation followed by testing levels, adjusting dosing as needed and again administering formulation. The frequency of dosing, testing, adjusting dosage and re-dosing can be determined by the caregiver as needed.

### The Current Theory Around the Homeostasis of Ca, PO₄, and PTH

Ca and PO₄ are extremely important minerals for maintaining healthy functions in human bodies. In mammals, the blood levels of Ca are strictly maintained in the range of 8.5˜11 mg/dL and those of phosphorus (P) in mature adults are in the range of 2.7˜4.5 mg/dL.

If a person is eating a normal diet, the expected amount of calcium in the urine is 100 to 300 mg/day and the normal level of phosphate in the urine is 900 to 1300 mg/day. Several health problems occur when the blood concentrations of these minerals move out of their normal ranges. For example, hypercalcemia (too high Ca levels) typically causes hyperactivity in neurons which sometimes causes epilepsy and in extreme cases of hypercalcemia causes comatosis or death. Excessive phosphate concentration is known to cause apoptosis of osteoblasts (bone forming cells) which impairs bone remodeling. Hyperphosphatemia (too high phosphate levels) is also known to typically cause blood vessel calcification by deposition of insoluble salts formed by excessive phosphate and calcium, which results in various cardiovascular and cerebrovascular diseases such as atherosclerosis, hypertension, heart failure, stroke, and so forth. Hypophosphatemia (abnormally low phosphate levels) impairs bone remodeling generally and causes growth retardation in younger patients who would normally still be growing.

According to the current theory of endocrinology that has been accepted for decades, endogenous hormones such as PTH, calcitriol, and calcitonin play key roles in regulating the homeostasis of Ca and PO₄. Among these, PTH has been considered as playing the central role in regulating homeostasis.

The primary function of PTH is to maintain Ca homeostasis in mammals. PTH stimulates active reabsorption of Ca from urine to serum at renal tubules after Ca has been once passively filtered at glomeruli. PTH binds its receptors expressed on renal tubule cells, upregulates protein kinases including protein kinase A (PKA) and accordingly upregulates cAMP in the cells, and increases Ca reabsorption. Reference ranges for PTH tests vary somewhat depending on the laboratory, and must be interpreted in association with calcium results. The following ranges are typical: Intact PTH: 10-65 pg/mL, PTH N-terminal (includes intact PTH): 8-24 pg/mL, PTH C-terminal (includes C-terminal, intact PTH, and midmolecule): 50-330 pg/mL.

PTH also affects osteoblasts through its receptors and PKA and other kinase mediated signaling cascades. In a physiological condition where the skeletal cells are consistently exposed to circulating PTH, the osteoblasts stimulated by PTH in turn stimulate osteoclasts to accelerate bone resorption. Overall, PTH accelerates the entire bone turnover when more Ca is released from the skeletal tissues into the circulation.

Combining these hormonal functions in renal tubules and skeletal tissues, PTH increases Ca blood levels.

Parathyroid glands have a mechanism for regulating PTH production levels. These glands express a sensor molecule that can detect the circulating levels of Ca, which is called a Ca receptor or Ca sensor. When high Ca levels are detected, parathyroid glands downregulate their PTH production. PTH production is increased when low Ca levels are detected. Thus, parathyroid glands regulate production and secretion of PTH based upon the circulating Ca levels and maintains Ca homeostasis.

PTH also contributes to PO₄ homeostasis. PTH is known to inhibit reabsorption of PO₄ at proximal tubules in the kidneys. PTH binds its receptors on the tubule cells, activates a PKA mediated cascade, reduces the amount and/or activities of sodium-dependent phosphate co-transporter (NaPi) on the tubule cells, and thereby inhibits PO₄ reabsorption. Namely, PTH reduces the serum levels of PO₄.

Although the detail mechanisms are yet to be understood, it seems that the parathyroid glands are capable of detecting circulating PO₄ levels in order to regulate their PTH production. When the circulating PO₄ levels remain elevated, the parathyroid glands produce more PTH, which should reduce serum PO₄ levels.

In summary, mutually regulating mechanisms exist between PTH and the minerals such as Ca and PO₄. Accordingly, methodologies for changing the physiological levels of these molecules must take these interconnected mechanisms into consideration.

### Current Methodologies for PTH Regulation

For the purpose of regulating Ca and PO₄ metabolisms, a few methods have been presented to date.

In accordance with one method synthetic molecules that modify the Ca receptors on parathyroid glands and act as agonists or antagonists have been developed. The agonists send a signal to parathyroid glands as if circulating Ca levels are high and thereby reduce PTH production. The antagonists send an opposite signal to increase PTH production.

Agonists are useful in treating conditions which results in excessive PTH secretion. Censapar, a Ca agonist, has been approved as a therapeutic to treat the secondary hyperparathyroidism in chronic kidney disease. Antagonists are being developed to treat bone loss because it has been known that a pulse-like stimulation of bone tissues with PTH promotes bone formation and that a pulse-like administration of a short half-life Ca antagonist might cause pulse-like production of PTH by the parathyroid glands.

Another method involves using an antibody selective to PTH. As such the antibody selectively neutralizes circulating PTH in order to treat hyperparathyroidism conditions.

In many cases, the ultimate benefits from PTH regulation is modifying Ca metabolism and/or bone turnover. However, because PTH also affects PO₄ metabolism, and PTH per se is regulated by Ca and PO₄, respectively, the currently available methodologies for regulating PTH are restricted by the currently understood mechanisms.

### Traditional Understandings of Ca and PO₄ Metabolisms

As described above, PTH increases Ca reabsorption and decreases PO₄ reabsorption in renal tubule thereby increasing serum levels of Ca and decreasing serum PO₄. PTH also recruits Ca from bone tissue to increase serum Ca levels. When PTH levels are extremely low, serum Ca levels are significantly reduced and reabsorption of PO₄ from the urine into the serum is increased for less inhibition by PTH. Thus, it is generally believed that Ca and PO₄ always move in opposite directions. In particular, it has been thought substantially impossible to simultaneously reduce serum levels of both Ca and PO₄. Further, controlling all three (Ca, PO₄, and PTH) simultaneously has not been considered as a possibility based on the current understandings of endocrinology.

### Phosphatonin—a Phosphate Regulating Hormone

There has been a hypothesis since the 80's that there may be one or more endogenous molecules that primarily regulates PO₄ metabolism. The virtual molecule was given a generic name, “phosphatonin” and several groups attempted to isolate the molecule.

A few novel molecules have been identified in the past few years and were found to regulate serum PO₄ levels. These molecules may be referred to as “phosphatonin,” (see U.S. Pat. No. 6,818,745) and consisted of MEPE, FGF-23, and FRP-4. All of them seemed to reduce the serum levels of PO₄ without affecting the serum levels of Ca or PTH.

All of these three molecules were identified from or correlated to rare diseases typically characterized by extremely low serum PO₄ levels, extremely low vitamin D₃ levels, and osteomalacia, but normal levels of serum Ca and PTH. These clinical observations in the diseases correlated to these candidate molecules of “phosphatonin” also strongly suggested that they are primary and selective regulators of PO₄ without affecting Ca or PTH.

Compared to the traditional understandings regarding the regulation of Ca, PO₄, and PTH, the discovery of “phosphatonin” appeared to be an advancement because it appeared to offer a method of controlling PO₄ without increasing Ca.

However, controlling Ca and PO₄ simultaneously or controlling all of Ca, PO₄, and PTH was yet to be achieved (see U.S. Pat. No. 6,673,900).

### Controlling PTH

One embodiment of the present invention discloses and describes a method for controlling serum levels of PTH. The method is characterized by administering to a subject a formulation comprised of an MEPE molecule once or a plurality of times within a short period of time e.g. 24 hours with measurements of PTH in a body fluid. The method or route of the administration can be either intravenous, subcutaneous, intraperitoneal, or other manner of injection, inhalation, nebulization, nasal spray, or other form of aerosols, or any other formulations for oral, topical, suppository and other administration route and measurements may be before, in between and after points of administration.

The patient being treated may be any mammals and the MEPE molecule can be a single sequence of a plurality of sequences chosen from (SEQ ID No. 1, 2, 4, 5, 7, 8, 10, or 12) or one of its functional fragments that comprises at least 51 consecutive amino acids which are biologically active and substantially equivalent to the amino acid sequence of the active full length molecule in terms of having phosphotonin activity. Any of these molecules can be pegylated, glycosylated and/or phosphorylated. The time and frequency of the injection can be any number, including but not limited to once, twice, or several times over 0-168 hour period. Measuring levels of all or any of Ca, PO₄ or PTH can be carried out before, during or after each or any of the points of administration. Administration of the MEPE molecule for much longer period than 168 hours to retain the serum levels of PTH for a longer period is also within the scope of this invention. The administered MEPE can be in a sustained release formulation to reduce the frequency of administration and reduced the frequency of taking measurements.

Methods are disclosed for controlling all or any of parathyroid hormone (PTH) levels, phosphate levels (PO₄) and calcium levels (Ca). The method comprises measuring all or any of the levels of the PTH, PO₄ and Ca in a patient and then administering to the patient a therapeutically effective amount of an amino acid sequence having phosphotonin activity. The sequence may have the SEQ ID NO:2, 3, 5, 6, or 8-13 and may comprise 51 or more amino acids. The steps of measuring and administering may be repeated any number of times over any period of time in order to carry out effective treatment of the patient.

Another aspect of the invention is a formulation manufactured for use in connection with a method such as described here including the specific method described above. The formulation may comprise a peptide with phosphotonin activity of the type described herein in combination with a carrier which carrier may be an injectable carrier or other type of carrier as described herein including an absorbable collagen sponge. Particular types of carriers may be chosen depending on the particular treatment being carried out on the patient. Formulations for use in carrying out particular methodologies are disclosed. Further, the manufacture of formulations for the use in carrying out particular methods of treatment are disclosed.

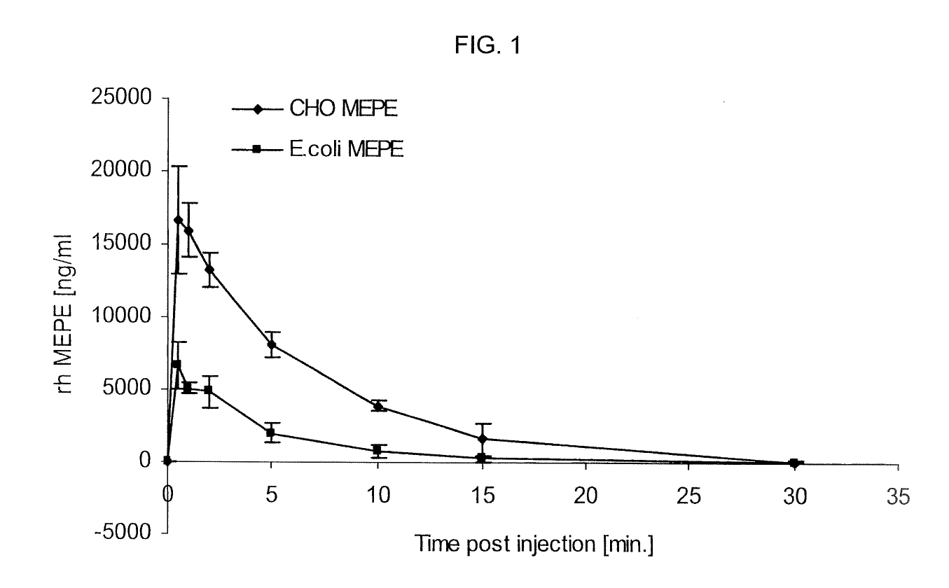

Example 1 and FIG. 1 show the pharmacokinetics of recombinant human MEPE (rhMEPE) made by genetically engineered E. coli or Chinese Hamster Ovarian (CHO) cells. As demonstrated, both E. coli and CHO-made rhMEPE showed relatively short retention in the circulation.

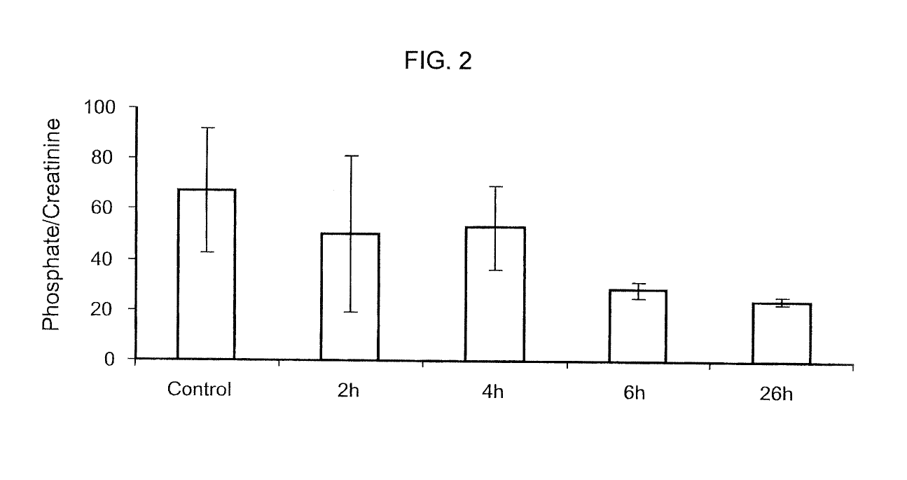

Example 2 and FIG. 2 exhibit the effect of rhMEPE on serum levels of PO₄. As already demonstrated in the prior art (U.S. Pat. No. 6,673,900, Bone 32 (2) 303-319, 2004), serum levels of PO₄ were reduced by the administration of rhMEPE to the rodents.

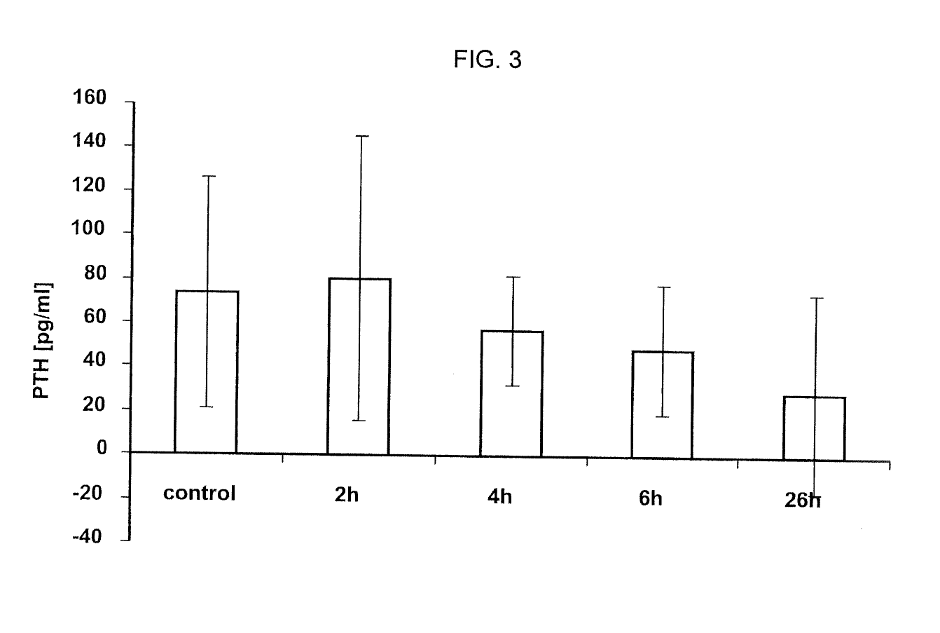

FIG. 3 in the same example (Example 2) show that the serum levels of PTH tend to be reduced by a plurality of bolus injections of the MEPE molecule by i.v. or i.p. route in a limited time such as four to 24 hours. Prior to these results PTH had been thought to be difficult to regulate. In particular, based on the fact that serum levels of PTH are generally normal in the patients of XLH or TIO tumor where MEPE is believed to be overproduced by bone or TIO tumor cells, this was a striking observation.

Previously MEPE was identified and cloned from a TIO tumor as a candidate of phosphatonin, which was believed to reduce serum PO₄ levels but not believed to affect PTH levels or specifically to reduce PTH levels.

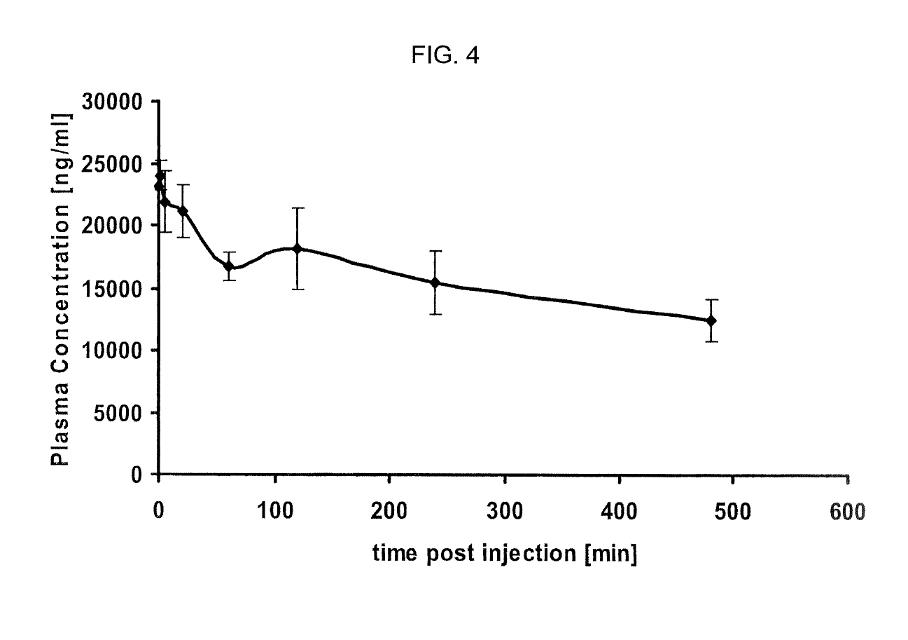

Further to verify this observation, a pegylated form of E. coli-produced rhMEPE (PEG-MEPE) was tested. Example 3 and FIG. 4 showed a very long half-life of PEG-MEPE. As compared to the E. coli or CHO-made rhMEPE which demonstrated approximately 3.5 minutes circulating half-life in the rats, the half-life of PEG-MEPE was extended to about eleven hours in the same model.

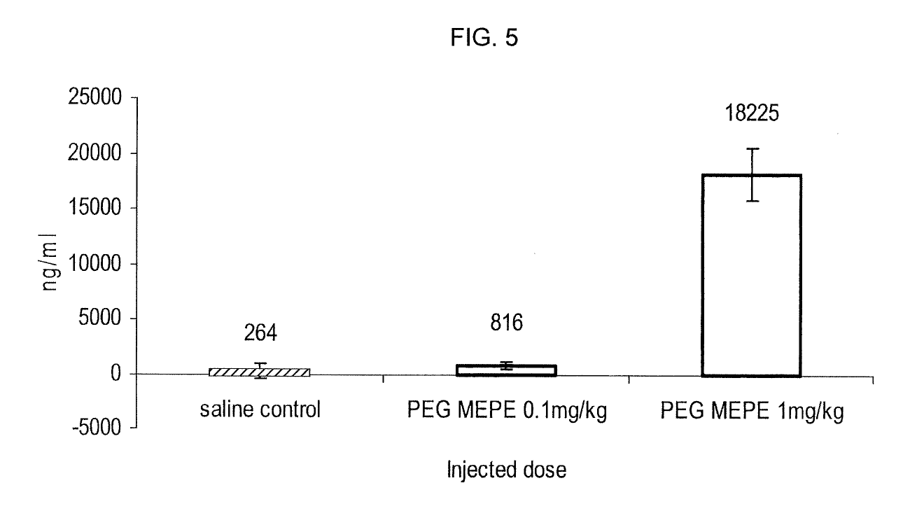

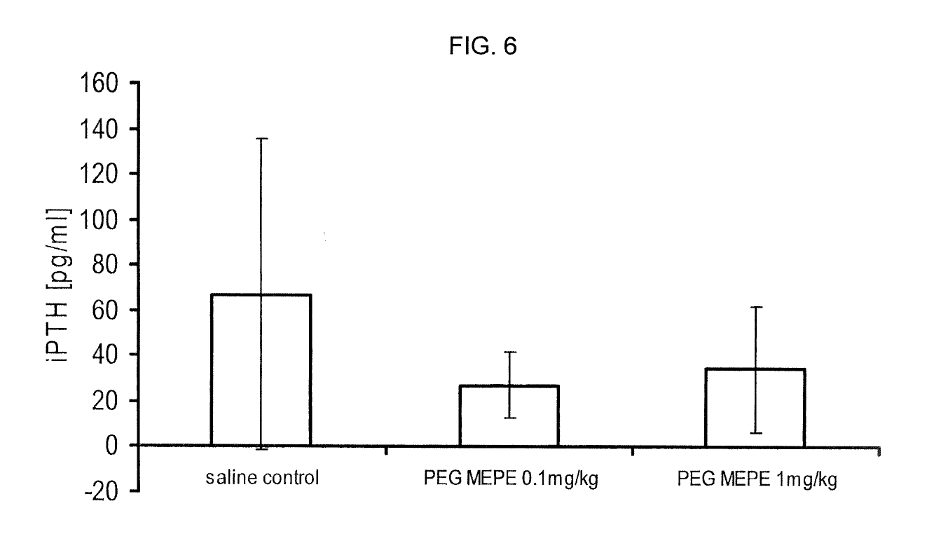

Thus, pegylation of rhMEPE enabled it to remain in the circulation for over 24 hours as exhibited in FIG. 5 in Example 4. As FIG. 6 in the same Example 4 indicates, the plasma PTH levels in the animals which received a single bolus injection of PEG-MEPE showed a tendency to reduce the plasma levels of PTH.

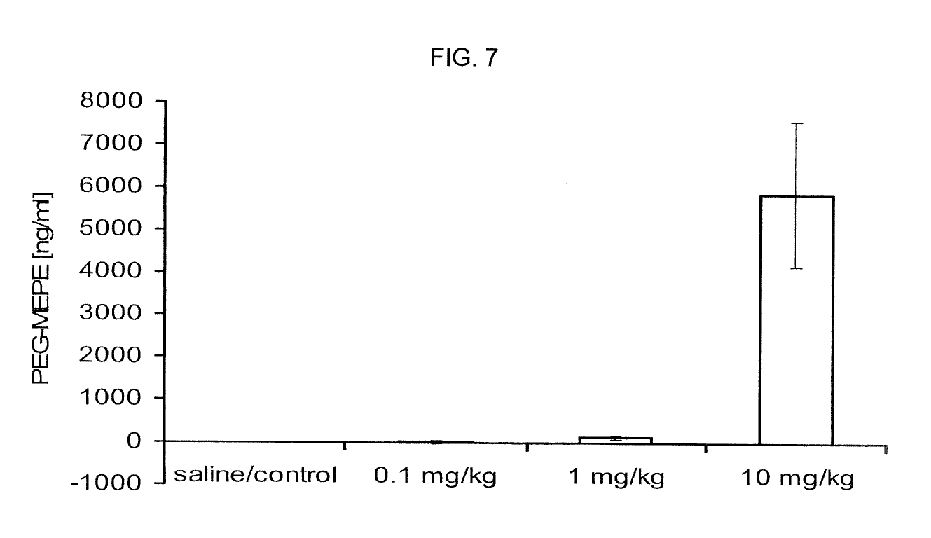

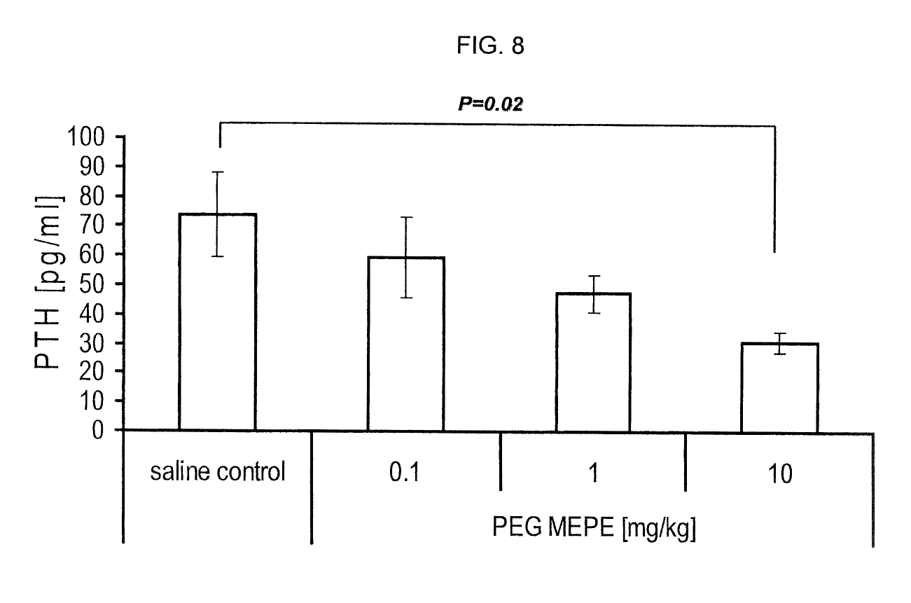

When the experiment period was extended to 72 hours as indicated by Example 5, the plasma levels of PTH clearly demonstrated a dose-dependent and statistically significant reduction as compared to the control (see FIG. 8). It was also confirmed the PEG-MEPE still remained in the circulation at 72 hours after a single bolus injection at 0 hour at its highest dose (see FIG. 7).

In summary, it was unexpectedly demonstrated for the first time that administration of the MEPE molecule to the animals was effective in reducing the circulating levels of PTH.

### Controlling Ca, PO₄, and PTH Simultaneously

In another embodiment of the invention a method is disclosed and described whereby serum levels of Ca and PO₄ are measured, and simultaneously reduced by administering a formulation comprised of an MEPE molecule once or a plurality of times (with intermittent measurements) within a short period of time e.g. less than 24 hours.

Although MEPE has been understood as “phosphatonin” and was known to reduce serum PO₄ levels it was not known to simultaneously reduce serum Ca levels.

There has been a general understanding in the field that the natural homeostasis of the body was that when one is elevated, the other is declined and that when one is declined, the other is elevated. Thus, the simultaneous reduction of the serum levels of both Ca and PO₄ is a novel and unexpected achievement. See Chapter 16 in the 5^{th} edition of “Primer on Metabolic Bone dieases and Disorders of Mineral Metabolism,” Mineral Balance and Homeostasis, by A E Broadus, pages 105-111, 2003. M J Favus Editor. Published by ASBMR, Washiungton D.C.

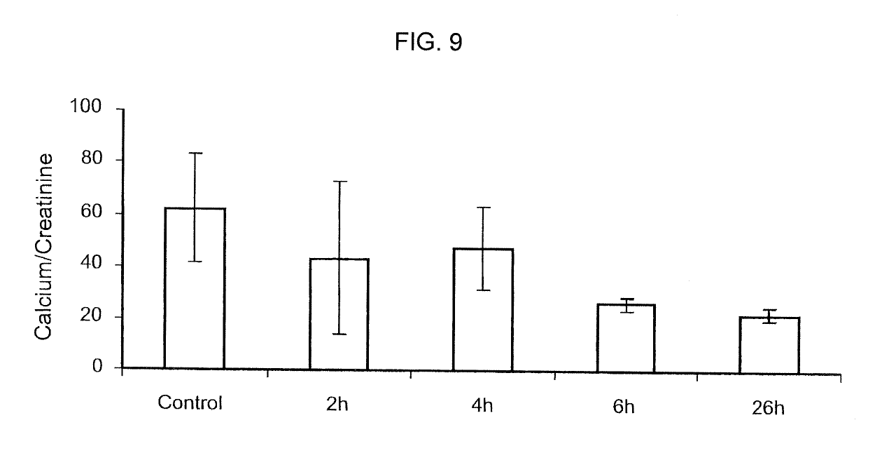

FIG. 9 that was a part of the results in Example 2 show a result whereby the serum levels of Ca were reduced as PTH levels were reduced (FIG. 3) by rhMEPE administration.

Because it has been known that one of the important biological functions of PTH was to regulate the serum levels of Ca, the observed reduction in the serum Ca levels in this experiment seemed natural as it followed the reduction of plasma PTH levels. However, the fact that the reduction of PTH and Ca occurred simultaneously with the reduction of the serum levels of PO₄ (FIG. 2) was a surprising observation as it has been believed to be extremely hard to achieve.

Combining the results indicated by FIGS. 2, 3, and 9, a simultaneous reduction in the circulating levels of Ca, PO₄, and PTH was achieved by administration of the MEPE molecule.

In addition, MEPE was found to inhibit sodium dependent phosphate co-transport in intestinal cells, which should have contributed to the reduction of serum PO₄ levels. Because the hypothesized activities of a “phosphatonin” were to control serum PO₄ levels by inhibiting renal PO₄ reabsorption, this intestinal activity of phosphatonin was also a new finding.

These observations suggest a novel mechanism of mineral and PTH homeostasis because sodium dependent phosphate co-transport should increase when PTH levels are decreased, because PTH is, in accordance with traditional theory, known to inhibit such transport.

Thus, while MEPE directly affects renal tubule cells reducing sodium dependent phosphate co-transport as its anticipated activities of “phosphatonin,” MEPE also appears to reduce the serum levels of PTH in a mechanism that is independent from its “phosphatonin” activities, and thereby reduce the serum levels of Ca, too.

### Methods of Treatment

This invention also relates to a method of treating patients suffering from metabolic imbalances of Ca, PO₄, and/or PTH as well as the subsequent clinical problems directly or indirectly caused by such imbalances.

Another embodiment of the present invention provides a method of treating hyperparathyroidism. The method is characterized by administering a formulation comprised of MEPE to the patients suffering from hyperparathyroidism.

In yet another embodiment there is disclosed a method of treating hyperphosphatemia and hyperparathyroidism simultaneously by administration of MEPE by reducing circulating PTH, Ca and PO₄ levels simultaneously. In another embodiment, there is disclosed a method of treating and/or preventing cardiovascular diseases by reducing Ca—P product in the blood by MEPE administration thereby reducing the excessive calcification of the blood vessels, which would benefit kidney patients significantly. Further, it was recently presented that PTH, together with Ca, plays an important role in increasing cardiovascular mortality. See Calcium, calcium regulatory hormones, and calcimimetics: impact on cardiovascular mortality. J Am Soc Nephrol. 2006 April; 17(4 Suppl 2):578-80. Administration of MEPE would improve such condition more globally.

In another method of the invention MEPE is administered to reduce all of PTH, Ca, and PO₄ in the serum simultaneously (ideal for the kidney patients), and obtain bone remodeling by incorporating Ca into the bone (to treat renal osteodystrophy and other bone diseases). All or any of these methods can be carried out with measuring levels of all or any of Ca, PO₄ and PTH and may further include adjusting dosing based on measurements made at various points in time. Thus, dosing, measuring, adjusting dosing and measuring can be repeated in any order and number of times over any desired period of treatment.

### Method of Administration

The method or route of the administration can be either intravenous, subcutaneous, intraperitoneal, intramuscular, intradermal, oral or topical. Oral administration may employ tablets, capsules, a syrup, elixir, or a sustained release composition. Topical administration may include a foam, gel, cream, ointment, transdermal patch, or paste. Suitable dosage forms are dependent upon the use or the route of entry. Formulations may be in suspensions, solutions or emulsions and may contain agents such as suspending, stabilizing and/or dispersing agents. Carriers or excipients can also be used to facilitate administration of the molecule. Examples of carriers include various sugars such as lactose, glucose, or sucrose, or types of starch, cellulose derivatives, gelatin, vegetable oils, polyethylene glycols and physiologically compatible solvents. A biologically active fragment comprised of 51 amino acids of any of SEQ ID NO:2, 3, 5, 6, 8-13 can be added to any of these carriers or to other carriers such as an absorabable collagen sponge (ACS) of any type including that ACS sold with rhBMP.

### Methods of Measuring Ca, PO₄ and PTH Levels

Calcium, PO₄, and PTH levels can be diagnosed by standard medical techniques, such as blood or urine analysis. For example, known methods for measuring calcium and phosphate ions in body fluids include titration, colorimetry, atomic absorptiometry, flame photometry, electrode method and enzyme methods. In addition, two tests are typically used to measure intact PTH and its terminal fragments. The C-terminal PTH assay is used to diagnose the ongoing changes in PTH metabolism that occur with secondary and tertiary hyperparathyroidism. The assay for intact PTH and the N-terminal fragment, which are both measured at the same time, is more accurate in detecting sudden changes in the PTH level. Representative methods for measuring calcium, PO₄, and PTH levels include but are not limited to, those described in U.S. Pat. Nos. 6,521,460; 6,387,646; and U.S. Application Nos: 20050191664; 20050130321; and 20030174802, as well as Liesener et al., Anal Bioanal Chem. 2005 August; 382(7): 1451-64; Clin Chim Acta. 2005 Jul. 1, 357(1):43-54; Clin Lab. 2005; 51(1-2):31-41; the disclosures of which are hereby incorporated by reference.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade and pressure is at or near atmospheric.

### Example 1

### Pharmacokinetic Profile of rhMEPE Produced by E. coli and by CHO Cells

Sprague-Dawley rats (˜300 g) were prepared by inserting femoral and jugular catheters for drug administration and blood collection respectively. Four rats were used for each type of material. rhMEPE was diluted in saline and injected (0.5 ml) to give a target dose of 1 mg/kg. Blood collected at 0, 0.5, 1, 2, 5, 10, 15 and 30 minutes. Blood was centrifuged to collect plasma and then frozen at −80 C until assay. Plasma levels of MEPE were determined using a competitive ELISA employing a rabbit polyclonal antibody made to a synthetic fragment of MEPE. Under these conditions, the ELISA has a linear detection range of ˜10 ng/ml to 1000 ng/ml). Samples from each rat were analyzed in duplicate and MEPE levels determined from a standard curve.

FIG. 1 demonstrates that both materials have a similar half life of approximately 3.5 minutes. However, the Cmax for the E. coli material was ˜6500 ng/ml whereas the Cmax for the CHO material was ˜16,500 ng/ml. As an indicator of total exposure, AUC was calculated and found to be ˜31,300 ng-min/ml for the E. coli and ˜115,400 ng-min/ml for the CHO material respectively.

### Example 2

### Effect of rhMEPE on Plasma Levels of Phosphate and Parathyroid Hormone (PTH)

Sprague Dawley rats (˜300 g) were injected three times with 2 mg/kg of E. coli produced rhMEPE at times 0, 2 hr, and 4 hr. Blood was collected prior to injection of MEPE (time 0) and then 2 hr post the first injection (2 hr time point), 2 hr post second injection (4 hr time point), 2 hr post third injection (6 hr time point) and finally at either 24 or 26 hr as indicated. Serum was collected and analyzed for creatinine, PO₄ and PTH. FIGS. 2 and 3 show the effects of rhMEPE on serum PO₄ when normalized to serum creatinine and PTH, respectively.

As shown in FIGS. 2 and 3, respectively, administration of rhMEPE results in a rapid reduction in both PO₄ and PTH component. In addition, the levels appear to remain depressed for at least 20 hrs following the last injection of rhMEPE.

### Example 3

### Pharmacokinetic Profile of E. coli rhMEPE Conjugated to Polyethylene Glycol (PEG)

rhMEPE was produced using an E. coli expressing system. The MEPE protein was then modified by the addition of PEG. The average molecular weight of the material used in this study was ˜130 kD. PEG-MEPE was diluted in saline and administered IV (via femoral catheter) to rats (˜300 g) at a dose of 1 mg/kg. A total of 4 rats were used in this study. Blood was then collected at various time points up to 4 hr post injecting and analyzed for MEPE using a competitive ELISA. FIG. 4 shows the plasma concentrations of MEPE over time following a single bolus injection of PEG-MEPE. From this study, it was determined that the half life for PEGE-MEPE was approximately 10.9 hrs. This is substantial enhancement compared to non-PEG MEPE which had a half life of approximately 3 minutes. From these data, we might expect a single administration of PEG-MEPE to maintain an enhanced biological response.

### Example 4

### Effect of PEG-MEPE on Plasma Levels of Parathyroid Hormone (PTH) at 24 Hour after the Injection

PEG-MEPE was prepared as described in Example 3. Rats (N=5/group) were injectable IV with either saline or 0.1 mg/kg or 1.0 mg/kg PEG-MEPE. Blood was collected 24 hr post injection and measured for MEPE levels using an ELISA (FIG. 5) or for plasma parathyroid hormone, PTH (FIG. 6). Administration of PEG-MEPE resulted in detectable levels of plasma MEPE approximately 3 and 70 times the level of the saline controls with doses of 0.1 and 1.0 mg/kg respectively. Plasma levels of PTH were measured in the same study (FIG. 6) and were found to be decreased 24 hrs following the PEG-MEPE administration. Thus, a single administration of PEG-MEPE is able to reduce PTH which in turn would lower serum calcium levels.

### Example 5

### Effect of PEG-MEPE on Plasma Levels of Parathyroid Hormone (PTH) at 72 Hour after the Injection

PEG-MEPE was prepared as described in Example 3. Rats (N=5/group) were injectable IV with either saline or 0.1 mg/kg, 1.0 mg/kg, or 10.0 mg/kg PEG-MEPE. Blood was collected 72 hr post injection and measured for MEPE levels using an ELISA (FIG. 7) or for plasma parathyroid hormone, PTH (FIG. 8). Administration of PEG-MEPE resulted in substantial levels of plasma MEPE in the high dose group and detectable levels in the 1 mg/kg group 72 hours post injection. Plasma levels of PTH were measured in the same study (FIG. 8) and were found to be decreased in a dose dependent manner 72 hrs following the PEG-MEPE administration. Thus, a single administration of PEG-MEPE results in a dose-dependent decrease in PTH 72 hrs later. It is of interest to note that even though the levels of plasma MEPE were not detectable in the low dose group and somewhat low in the mid-dose group, there was still a decrease in PTH levels. Thus, it appears as if the effect on PTH levels can persist after much of the MEPE has been cleared from circulation.

### Example 6

### Effect of rhMEPE on Plasma Levels of Calcium

In the same experiment as the one in Example 2, blood was collected at the same schedule and the serum was analyzed for Ca, too. FIG. 9 shows the effects of rhMEPE on serum Ca+ when normalized to serum creatinine.

As shown in FIG. 9, administration of rhMEPE results in a rapid reduction in serum Ca. In addition, the levels appear to remain depressed for at least 20 hrs following the last injection of MEPE.

In combining with the results from the same experiment as the one in Example 2, it was demonstrated that injection of rhMEPE reduced all three elements, i.e., PO₄, PTH, and Ca simultaneously.

### Sequence Listing

For full length sequences see U.S. Pat. No. 6,673,900 issued Jan. 6, 2004 which is incorporated herein by reference in its entirety as are the patents and publications cited therein along with subsequent publications of Peter Rowe and see U.S. Pat. No. 6,911,425 issued Jun. 26, 2005 which is incorporated herein by reference in its entirety as are the patents and publications cited therein.

Full 525 amino acid sequence of human MEPE—2 Variants (SEQ ID No. 1)

509 amino acid sequence of human MEPE after cleaving off 16 amino acid signal sequence from its full length—2 Variants like SEQ ID No. 1 (SEQ ID No. 2)

430 amino acid sequence from the C-terminus of human MEPE—2 Variants like SEQ ID No. 1 (SEQ ID No. 3)

Full amino acid sequence of macaque MEPE (SEQ ID No. 4)

Macaque MEPE after cleaving off the signal sequence (SEQ ID No. 5)

C-terminus portion of macaque MEPE corresponding to SEQ ID No. 3 in human MEPE (SEQ ID No. 6)

Full amino acid sequence of canine MEPE (SEQ ID No. 7)

Canine MEPE after cleaving off the signal sequence (SEQ ID No. 8)

C-terminus portion of canine MEPE corresponding to SEQ ID No. 3 in human MEPE (SEQ ID No. 9)

Full amino acid sequence of rat MEPE (SEQ ID No. 10)

Rat MEPE after cleaving off the signal sequence (SEQ ID No. 11)

Full amino acid sequence of mouse MEPE (SEQ ID No. 12)

Mouse MEPE after cleaving off the signal sequence (SEQ ID No. 13)

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1-42. (canceled)

43. A method of treating bone loss, comprising:
diagnosing a subject as suffering from bone loss due to renal osteodystrophy;
injecting the subject diagnosed with bone loss with a therapeutically effective amount of a formulation comprised of a carrier and a molecule chosen from amino acid sequences-comprising SEQ ID No. 2, 3, 5, 6, and 8-13.

44. The method of claim 43, wherein the formulation is injected in amounts and over a period of time so as to have an additional effect chosen from:
(a) reducing circulating levels of phosphate;
(b) reducing effects of sodium dependent phosphate co-transporter in renal tubule cells;
(c) reducing intestinal absorption of phosphate;
(d) reducing effects of sodium dependent phosphate co-transporter in intestinal cells; and
(e) absorbing phosphate in the patient's circulation into the patient's hard tissues.

45. The method of claim 44, wherein all of (a)-(e) are obtained.

46. A method of reducing circulating levels of parathyroid hormone, comprising:
diagnosing the subject as suffering from elevated levels of parathyroid hormone due to renal osteodystrophy;
injecting the subject diagnosed with elevated levels of parathyroid hormone with a therapeutically effective amount of a formulation comprised of a carrier and a molecule chosen from amino acid sequences-comprising SEQ ID No. 2, 3, 5, 6, and 8-13.

47. The method of claim 46, wherein the formulation is injected in amounts and over a period of time so as to have an additional effect chosen from:
(a) reducing circulating levels of phosphate;
(b) reducing effects of sodium dependent phosphate co-transporter in renal tubule cells;
(c) reducing intestinal absorption of phosphate;
(d) reducing effects of sodium dependent phosphate co-transporter in intestinal cells; and
(e) absorbing phosphate in the patient's circulation into the patient's hard tissues.

48. The method of claim 47, wherein all of (a)-(e) are obtained.


SEQUENCE LISTING (13 sequences)

SEQ ID NO: 1 - protein, 525 aa, Homo sapiens
MRVFCVGLLLFSVTWAAPTFQPQTEKTKQSCVEEQRQEEKNKDNIGFHHLGKRINQELSSKENIVQERKKDLSLSEASENKGSSKSQNYFTNRQRLNKEYSISNKENTHNGLRMSIYPKSTGNKGFEDGDDAISKLHDQEEYGAALIRNNMQHIMGPVTAIKLLGEENKENTPRNVLNIIPASMNYAKAHSKDKKKPQRDSQAQKSPVKSKSTHRIQHNIDYLKHLSKVKKIPSDFEGSGYTDLQERGDNDISPFSGDGQPFKDIPGKGEATGPDLEGKDIQTGFAGPSEAESTHLDTKKPGYNEIPEREENGGNTIGTRDETAKEADAVDVSLVEGSNDIMGSTNFKELPGREGNRVDAGSQNAHQGKVEFHYPPAPSKEKRKEGSSDAAESTNYNEIPKNGKGSTRKGVDHSNRNQATLNEKQRFPSKGKSQGLPIPSRGLDNEIKNEMDSFNGPSHENIITHGRKYHYVPHRQNNSTRNKGMPQGKGSWGRQPHSNRRFSSRRRDDSSESSDSGSSSESDGD

SEQ ID NO: 2 - protein, 509 aa, Homo sapiens
APTFQPQTEKTKQSCVEEQRQEEKNKDNIGFHHLGKRINQELSSKENIVQERKKDLSLSEASENKGSSKSQNYFTNRQRLNKEYSISNKENTHNGLRMSIYPKSTGNKGFEDGDDAISKLHDQEEYGAALIRNNMQHIMGPVTAIKLLGEENKENTPRNVLNIIPASMNYAKAHSKDKKKPQRDSQAQKSPVKSKSTHRIQHNIDYLKHLSKVKKIPSDFEGSGYTDLQERGDNDISPFSGDGQPFKDIPGKGEATGPDLEGKDIQTGFAGPSEAESTHLDTKKPGYNEIPEREENGGNTIGTRDETAKEADAVDVSLVEGSNDIMGSTNFKELPGREGNRVDAGSQNAHQGKVEFHYPPAPSKEKRKEGSSDAAESTNYNEIPKNGKGSTRKGVDHSNRNQATLNEKQRFPSKGKSQGLPIPSRGLDNEIKNEMDSFNGPSHENIITHGRKYHYVPHRQNNSTRNKGMPQGKGSWGRQPHSNRRFSSRRRDDSSESSDSGSSSESDGD

SEQ ID NO: 3 - protein, 430 aa, Homo sapiens
LNKEYSISNKENTHNGLRMSIYPKSTGNKGFEDGDDAISKLHDQEEYGAALIRNNMQHIMGPVTAIKLLGEENKENTPRNVLNIIPASMNYAKAHSKDKKKPQRDSQAQKSPVKSKSTHRIQHNIDYLKHLSKVKKIPSDFEGSGYTDLQERGDNDISPFSGDGQPFKDIPGKGEATGPDLEGKDIQTGFAGPSEAESTHLDTKKPGYNEIPEREENGGNTIGTRDETAKEADAVDVSLVEGSNDIMGSTNFKELPGREGNRVDAGSQNAHQGKVEFHYPPAPSKEKRKEGSSDAAESTNYNEIPKNGKGSTRKGVDHSNRNQATLNEKQRFPSKGKSQGLPIPSRGLDNEIKNEMDSFNGPSHENIITHGRKYHYVPHRQNNSTRNKGMPQGKGSWGRQPHSNRRFSSRRRDDSSESSDSGSSSESDGD

SEQ ID NO: 4 - protein, 555 aa, Macaca mulatta
MRVFCVGLLFLSVTWAAPTFQPQTEKTKQSCVEEQRITYKGHHEKHGHYVFKCVYMSPGKKNQTDVKQEEKNKDNIGLHHLGKRRYQELSSKENIVQERKKDLSLSEASENNGSSKSQNYFTNRQRLNKEYSISNKENIHNGLRMSIYPKSTGNKQFADGDDAISKLHDQEEYGAALIRNNMQHIMGPVTAIKLLGEENKQSKPKNVLNKIPASMNYAKAHSKDKKKPQRDSQVQKVPVKSKSTHRTQHNIDYPKHLSKVKKIPSDFEGSGYTDLQERGDNDMSPFSGDGQPFKDIPDKGEATGPDLEGKDIQTGFAGPSEAESTNLDTKEPGYNEIPEREENGGNTIGTGDETAKEADAVNVSLVEGNNDIMGSTNFKELPGREGNRVDVGGQNAHQGKVEFHYPPAPSKEKRKEGSSDATESTNYNEIPKNDKGSARKGVDDSNRNQAILHEKQRFPSKGKSQGLPIPSRGLDNEIKTEMDSLNGPSNENIPHSRKYHYVPHRQNNPTRNKGMPHGKGSWGRQPYSNRRLSSRRREDSSESSDSGSSSESDGD

SEQ ID NO: 5 - protein, 539 aa, Macaca mulatta
APTFQPQTEKTKQSCVEEQRITYKGHHEKHGHYVFKCVYMSPGKKNQTDVKQEEKNKDNIGLHHLGKRRYQELSSKENIVQERKKDLSLSEASENNGSSKSQNYFTNRQRLNKEYSISNKENIHNGLRMSIYPKSTGNKQFADGDDAISKLHDQEEYGAALIRNNMQHIMGPVTAIKLLGEENKQSKPKNVLNKIPASMNYAKAHSKDKKKPQRDSQVQKVPVKSKSTHRTQHNIDYPKHLSKVKKIPSDFEGSGYTDLQERGDNDMSPFSGDGQPFKDIPDKGEATGPDLEGKDIQTGFAGPSEAESTNLDTKEPGYNEIPEREENGGNTIGTGDETAKEADAVNVSLVEGNNDIMGSTNFKELPGREGNRVDVGGQNAHQGKVEFHYPPAPSKEKRKEGSSDATESTNYNEIPKNDKGSARKGVDDSNRNQAILHEKQRFPSKGKSQGLPIPSRGLDNEIKTEMDSLNGPSNENIPHSRKYHYVPHRQNNPTRNKGMPHGKGSWGRQPYSNRRLSSRRREDSSESSDSGSSSESDGD

SEQ ID NO: 6 - protein, 429 aa, Macaca mulatta
LNKEYSISNKENIHNGLRMSIYPKSTGNKQFADGDDAISKLHDQEEYGAALIRNNMQHIMGPVTAIKLLGEENKQSKPKNVLNKIPASMNYAKAHSKDKKKPQRDSQVQKVPVKSKSTHRTQHNIDYPKHLSKVKKIPSDFEGSGYTDLQERGDNDMSPFSGDGQPFKDIPDKGEATGPDLEGKDIQTGFAGPSEAESTNLDTKEPGYNEIPEREENGGNTIGTGDETAKEADAVNVSLVEGNNDIMGSTNFKELPGREGNRVDVGGQNAHQGKVEFHYPPAPSKEKRKEGSSDATESTNYNEIPKNDKGSARKGVDDSNRNQAILHEKQRFPSKGKSQGLPIPSRGLDNEIKTEMDSLNGPSNENIPHSRKYHYVPHRQNNPTRNKGMPHGKGSWGRQPYSNRRLSSRRREDSSESSDSGSSSESDGD

SEQ ID NO: 7 - protein, 643 aa, Canis lupus
MCLQVFTRKEPPSRKICPETANPVEEAKYSEGKIYQRVSEMQTVCLGLLLFSVTWAAPQACSKILRSSSAGSPVTPKFKPKSLEHAARIYSFLVSDLSPLQESSCLSSLEKIDLQACRKERDKITYKGHHEKHGYYIFKYVYTSSGRKNQTDIKQEEKNKDNTALQNSGKRRNQEPAPKENIAQEREKNLSILGASENKESSKTQTLFENIQTTNEVDSINNKENAHSDLKMSVYLESPGNNGAEDKDNAINKSHDQEEYGTALIRNNMQHIMEPGTVTELLAEENKENKPRNILSKIPASVNYVKVPSKNRKNYQRDPQAQNIPVKSKSTHHTQHNIDYPKQLQKVKKIPSDFEGSGYPDLQERGDNDISPFSGDGQPFKDISGKGEPIGPDLKGTDIQTESSGPREADTISPDARRPGYNEIPEREESGGSTTGTRDETRKEASTDVSLVEGSNDIIGSTNFKELPGKEGNRVGANSQNAHQGKVEFHYPFPPSKEKKAEGSSDVAESTNYNEIPKNGKGSSRKDTEHSNRNKVISTGKQRFPTKGKSQSQLVPSHDLDNEIKNEIGSHNGLNNEETIITQSRKNHYVPHRQNSTWNKGVPKRKGSWGYRKPHSRRRVSPPRRHDSSESSDSDSSSESDGD

SEQ ID NO: 8 - protein, 623 aa, Canis lupus
ANPVEEAKYSEGKIYQRVSEMQTVCLGLLLFSVTWAAPQACSKILRSSSAGSPVTPKFKPKSLEHAARIYSFLVSDLSPLQESSCLSSLEKIDLQACRKERDKITYKGHHEKHGYYIFKYVYTSSGRKNQTDIKQEEKNKDNTALQNSGKRRNQEPAPKENIAQEREKNLSILGASENKESSKTQTLFENIQTTNEVDSINNKENAHSDLKMSVYLESPGNNGAEDKDNAINKSHDQEEYGTALIRNNMQHIMEPGTVTELLAEENKENKPRNILSKIPASVNYVKVPSKNRKNYQRDPQAQNIPVKSKSTHHTQHNIDYPKQLQKVKKIPSDFEGSGYPDLQERGDNDISPFSGDGQPFKDISGKGEPIGPDLKGTDIQTESSGPREADTISPDARRPGYNEIPEREESGGSTTGTRDETRKEASTDVSLVEGSNDIIGSTNFKELPGKEGNRVGANSQNAHQGKVEFHYPFPPSKEKKAEGSSDVAESTNYNEIPKNGKGSSRKDTEHSNRNKVISTGKQRFPTKGKSQSQLVPSHDLDNEIKNEIGSHNGLNNEETIITQSRKNHYVPHRQNSTWNKGVPKRKGSWGYRKPHSRRRVSPPRRHDSSESSDSDSSSESDGD

SEQ ID NO: 9 - protein, 430 aa, Canis lupus
TNEVDSINNKENAHSDLKMSVYLESPGNNGAEDKDNAINKSHDQEEYGTALIRNNMQHIMEPGTVTELLAEENKENKPRNILSKIPASVNYVKVPSKNRKNYQRDPQAQNIPVKSKSTHHTQHNIDYPKQLQKVKKIPSDFEGSGYPDLQERGDNDISPFSGDGQPFKDISGKGEPIGPDLKGTDIQTESSGPREADTISPDARRPGYNEIPEREESGGSTTGTRDETRKEASTDVSLVEGSNDIIGSTNFKELPGKEGNRVGANSQNAHQGKVEFHYPFPPSKEKKAEGSSDVAESTNYNEIPKNGKGSSRKDTEHSNRNKVISTGKQRFPTKGKSQSQLVPSHDLDNEIKNEIGSHNGLNNEETIITQSRKNHYVPHRQNSTWNKGVPKRKGSWGYRKPHSRRRVSPPRRHDSSESSDSDSSSESDGD

SEQ ID NO: 10 - protein, 435 aa, Rattus norvegicus
MQAVSVGLFLFSMTWAAPKLNEDGSSGGNQGNIHLASVKPEPMVGKGTEGGRDAPLHLLDQNRQGATLLRNITQPVKSLVTGTEVQSDRNKEKKPQSVLSVIPTDVHNTNDYSEDTENQQRDLLLQNSPGQSKHTPRARRSTHYLTHLPQIRKILSDFEDSASPDLLVRGDNDVPPFSGDGQHFMHTPDRGGAVGSDPESSAGHPVSGSSNVEIVDPHTNGLGSNEIPGREGHIGGAYATRGKTAQGAGSADVSLVEGSNEITGSTKFRELPGKEGNRVDASSQNAHQGKVEFHYPQAPSKEKVKGGSREHTGKAGYNEIPKSSKGGASKDAEESKGNQVTLTESQRFPGKGKGQSSHSLGNEVKSEEDSSNSLSREGIAIAHRRTSHPTRNRGMSQRRGSWASRRPHPHRRVSTRQRDSSESSSSGSSSESSGD

SEQ ID NO: 11 - protein, 417 aa, Rattus norvegicus
KLNEDGSSGGNQGNIHLASVKPEPMVGKGTEGGRDAPLHLLDQNRQGATLLRNITQPVKSLVTGTEVQSDRNKEKKPQSVLSVIPTDVHNTNDYSEDTENQQRDLLLQNSPGQSKHTPRARRSTHYLTHLPQIRKILSDFEDSASPDLLVRGDNDVPPFSGDGQHFMHTPDRGGAVGSDPESSAGHPVSGSSNVEIVDPHTNGLGSNEIPGREGHIGGAYATRGKTAQGAGSADVSLVEGSNEITGSTKFRELPGKEGNRVDASSQNAHQGKVEFHYPQAPSKEKVKGGSREHTGKAGYNEIPKSSKGGASKDAEESKGNQVTLTESQRFPGKGKGQSSHSLGNEVKSEEDSSNSLSREGIAIAHRRTSHPTRNRGMSQRRGSWASRRPHPHRRVSTRQRDSSESSSSGSSSESSGD

SEQ ID NO: 12 - protein, 433 aa, Mus musculus
MQAVSVGLLLFSMTWAAPMPNEDRSSCGNQDSIHKDLAASVYPDPTVDEGTEDGQGALLHPPGQDRYGAALLRNITQPVKSLVTGAELRREGNQEKRPQSVLSVIPADVNDAKVSLKDIKNQESYLLTQSSPVKSKHTKHTRQTRRSTHYLTHLPQIKKTPSDLEGSGSPDLLVRGDNDVPPFSGDGQHFMHIPGKGGAGSGPESSTSRPLSGSSKAEVIDPHMSGLGSNEIPGREGHGGSAYATRDKAAQGAGSAGGSLVGGSNEITGSTNFRELPGKEGNRINAGSQNAHQGKVEFHYPQVASREKVKGGVEHAGRAGYNEIPKSSKGSSSKDAEESKGNQLTLTASQRFPGKGKSQGPALPSHSLSNEVKSEENHYVFHGQNNLTPNKGMSQRRGSWPSRRPNSHRRASTRQRDSSESSSSGSSSESHGD

SEQ ID NO: 13 - protein, 415 aa, Mus musculus
MPNEDRSSCGNQDSIHKDLAASVYPDPTVDEGTEDGQGALLHPPGQDRYGAALLRNITQPVKSLVTGAELRREGNQEKRPQSVLSVIPADVNDAKVSLKDIKNQESYLLTQSSPVKSKHTKHTRQTRRSTHYLTHLPQIKKTPSDLEGSGSPDLLVRGDNDVPPFSGDGQHFMHIPGKGGAGSGPESSTSRPLSGSSKAEVIDPHMSGLGSNEIPGREGHGGSAYATRDKAAQGAGSAGGSLVGGSNEITGSTNFRELPGKEGNRINAGSQNAHQGKVEFHYPQVASREKVKGGVEHAGRAGYNEIPKSSKGSSSKDAEESKGNQLTLTASQRFPGKGKSQGPALPSHSLSNEVKSEENHYVFHGQNNLTPNKGMSQRRGSWPSRRPNSHRRASTRQRDSSESSSSGSSSESHGD